(19) United States
(12) Patent Application Publication
(10) Pub. No.: US 2012/0004733 A1
(43) Pub. Date: Jan. 5, 2012

(54) MODULAR ARTICULATING PROSTHESES AND ASSOCIATED METHODS

(76) Inventors: Brian C. Hodorek, Winona Lake, IN (US); Robert Courtney, JR., Pierceton, IN (US)

(21) Appl. No.: 12/827,442

(22) Filed: Jun. 30, 2010

(51) Int. Cl. A61F 2/40; B23P 11/00

(52) U.S. Cl. 623/19.11; 29/428

(57) ABSTRACT

Joint prostheses and associated methods that have a medialized center of rotation, inhibit subluxation of the implant while facilitating full range of motion and normal articular function, are able to be implanted using standard bone preparation techniques, and/or provide increased implant lifetime.

### BACKGROUND

A standard shoulder joint prosthesis includes an artificial ball-and-socket joint with the ball portion replacing the humeral head and the socket portion implanted in the glenoid cavity of the scapula. Generally, this type of arrangement is appropriate where the rotator cuff is relatively intact and functional for stabilizing the implant. The reverse arrangement—the ball portion secured to the scapula and the socket portion secured to the humeral head—is termed a “reverse shoulder prosthesis” and is often used where the rotator cuff of the patient is relatively less functional. In both the standard and reverse configurations, however, long term loosening of the muscles supporting the prosthesis is a concern. For example, a common failure mode of a reverse shoulder prosthesis is continued degradation of the deltoid muscle, which eventually allows the prosthesis to sublux, or separate, thereby interfering with proper functioning of the joint.

### SUMMARY

Some embodiments relate to joint prostheses and associated methods that have a medialized center of rotation, inhibit subluxation of the implant while facilitating full range of motion and normal articular function, are able to be implanted using standard bone preparation techniques, and/or provide increased implant lifetime.

Some embodiments relate to a joint prosthesis adapted to be secured to a first bone and a second bone for facilitating relative articulation between the first and second bones. The joint prosthesis includes a first articulation component defining a first articulation surface that is substantially convex and a second articulation component defining a second articulation surface that is substantially concave and a third articulation surface that is substantially convex. The first articulation surface of the first articulation component is engaged with the second articulation surface of the second articulation component such that the first articulation component is substantially limited in angulation relative to the second articulation component within a first plane. The prosthesis also includes a third articulation component defining a fourth articulation surface that is substantially concave, the third articulation surface of the second articulation component being engaged with the fourth articulation surface of the third articulation component such that the third articulation component is substantially limited in angulation relative to the first articulation component within a second plane that is angularly offset from the first plane.

Other embodiments relate to a virtual ball-and-socket prosthesis for replacing a joint between a first bone and a second bone. The prosthesis includes means for limiting angular articulation of a first articulation component in sliding contact with a second articulation component to changes in pitch and means for limiting angular articulation of a third articulation component in sliding contact with the second articulation component to changes in yaw. The prosthesis also includes first bone anchor means for securing the first articulation component to a first bone and second bone anchor means for securing the third articulation component to a second bone, as well as means for allowing changes in roll between the first bone anchor means and the second bone anchor means.

Some embodiments relate to a virtual ball-and-socket prosthesis for replacing a natural joint between two bones. The prosthesis includes a first bone anchor component, a second bone anchor component, and a plurality of articulation components that articulatably join the first and second bone anchor components, the plurality of articulation components defining a pitch bearing interface, a yaw bearing interface separate from the pitch bearing interface, and a roll bearing interface separate from both the pitch and yaw bearing interfaces. The plurality of articulation components are secured relative to one another such that articulation between the first and second bone anchors in pitch is borne by the pitch bearing surface, articulation between the first and second bone anchors in yaw is borne by the yaw bearing interface, and medial rotational articulation between the first and second bone anchors is borne by the rotational bearing interface.

Still other embodiments relate to a method of assembling an artificial joint between bones. The method includes securing a first articulation component having a first articulation surface that is convex to a second articulation component having a second articulation surface that is concave such that the first articulation surface of the first articulation component is engaged with the second articulation surface of the second articulation component and the first articulation component is limited in angulation relative to the second articulation component to a first plane. The method also includes securing a third articulation component defining a fourth articulation surface that is concave to the second articulation component such that a third articulation surface of the second articulation component that is convex is engaged with the fourth articulation surface of the third articulation component and the third articulation component is limited in lateral angulation relative to the first articulation component to a second plane that is angularly offset from the first plane.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of a first joint prosthesis, according to some embodiments.

FIG. 2 is a perspective view of the joint prosthesis of FIG. 1 in an unassembled state, according to some embodiments.

FIGS. 3 and 4 show a first articulation component of the joint prosthesis of FIG. 1, according to some embodiments.

FIGS. 5 and 6 show a second articulation component of the joint prosthesis of FIG. 1, according to some embodiments.

FIGS. 7 and 8 show a third articulation component of the joint prosthesis of FIG. 1, according to some embodiments.

FIG. 9 is a sectional view of the joint prosthesis of FIG. 1, according to some embodiments.

FIG. 10 is a perspective view of a second joint prosthesis, according to some embodiments.

FIG. 11 is a cutaway view of the joint prosthesis of FIG. 10, according to some embodiments.

FIG. 12 is a perspective view of a first articulation component of the joint prosthesis of FIG. 10, according to some embodiments.

FIG. 13 is a perspective view of a second articulation component of the joint prosthesis of FIG. 10.

FIG. 14 is a perspective view of a third articulation component of the joint prosthesis of FIG. 10.

While the invention is amenable to various modifications, permutations, and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

As described in greater detail, some embodiments relate to an artificial, virtual ball-and-socket joint that includes a linked articulation assembly adapted to reduce and/or prevent subluxation of the artificial joint due, for example, to relaxation of the muscles supporting the artificial joint. Additionally, in some implementations, the linked articulation assembly of the artificial joint includes a plurality of distinct articulation interfaces for bearing movement of the artificial joint in distinct coordinate directions, such as a first interface for supporting articulation along the anteroposterior direction, a second interface for supporting articulation along the inferosuperior direction, and one or more interfaces for supporting rotational articulation. The separate interfaces provide means for reducing wear, as the bearing surfaces need only support movement along one discrete direction, which can be contrasted to the bearing surfaces of a typical ball-and-socket joint. While various features associated with some embodiments have been described above, it should be understood that various additional or alternate features are contemplated.

The terms pitch, roll, and yaw are also used, where roll generally refers to angulation, or rotation, in a first plane through which a longitudinal axis of a body orthogonally passes (e.g., rotation about a longitudinal axis passing through the glenoid), pitch refers to angulation, or rotation, in a second plane orthogonal to the first plane, and yaw refers to angulation, or rotation, in a third plane orthogonal to the first and second planes. In some embodiments, pitch is angulation in the anteroposterior direction, yaw is angulation in the inferosuperior direction, and roll is medial rotational articulation.

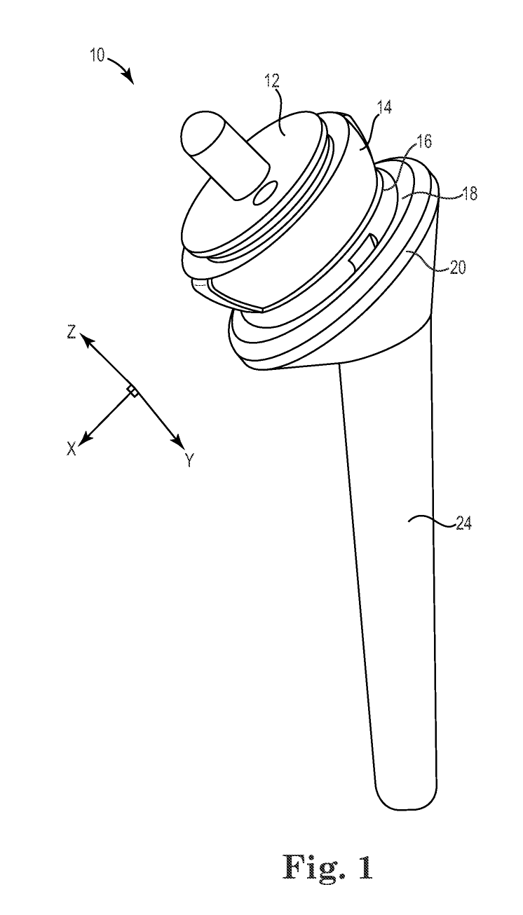

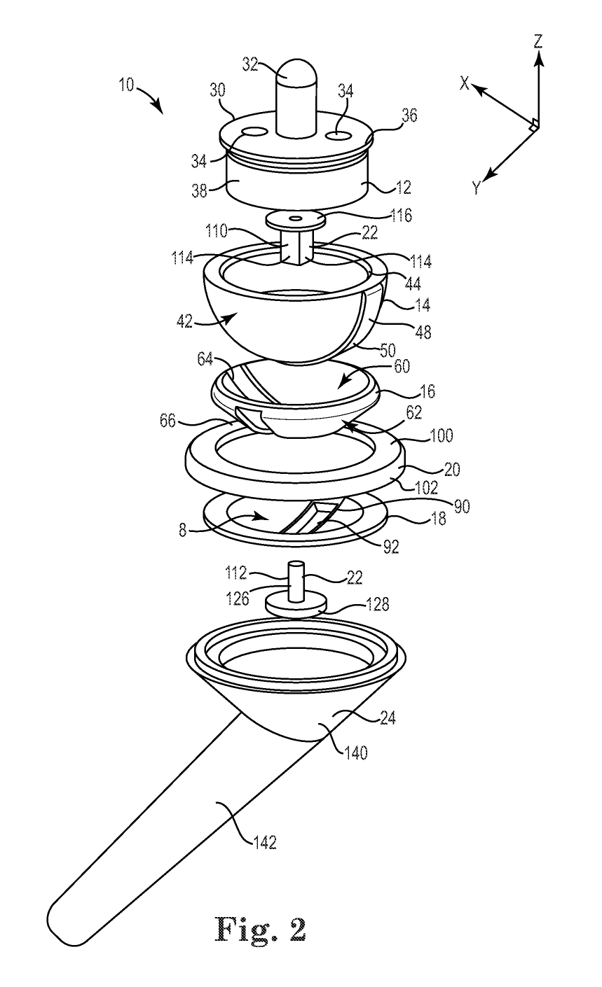

FIG. 1 shows a joint prosthesis 10 (also described as an artificial joint or a prosthesis) in an assembled state and FIG. 2 shows the joint prosthesis 10 in an unassembled state, according to some embodiments. As shown in FIGS. 1 and 2, the joint prosthesis 10 is adapted as a reverse shoulder prosthesis for replacing the gleno-humeral joint of a patient. Though the joint prosthesis 10 is adapted as a reverse shoulder prosthesis according to some embodiments, in other embodiments the joint prosthesis 10 is adapted as a traditional shoulder prosthesis or as a prosthesis for other bodily joints, such as the hip, for example.

As shown, the prosthesis 10 includes a first bone anchor 12 (also described as a base plate or an articulation component), a first articulation component 14 (also described as a glenosphere), a second articulation component 16 (also described as a liner or a disk), a third articulation component 18 (also described as a rotational plate), a locking ring 20 (also described as a locking member), a peg 22 (also described as a fastener, a guide, or a locking bolt), and a second bone anchor 24 (also described as a stem). The prosthesis 10 is generally adapted as a virtual ball-and-socket joint, being able to articulate through a wide range of motion similar to that of a traditional ball-and-socket joint by supporting freedom of movement between the first and second bone anchors 12, 24 in at least three coordinate directions, such as X-, Y-, and Z-axis angular articulation. For example, the prosthesis 10 optionally facilitates angular articulation relative to the X-axis (also described as pitch), or parallel to the Y-Z plane, relative to the Y-axis (also described as yaw), or parallel to the X-Z plane, and relative to the Z-axis (also described as roll), or parallel to the X-Y plane. In some embodiments, angular articulation relative to the X-axis corresponds to front-back motion or anteroposterior articulation, angular articulation relative to the Y-axis corresponds to up-down motion or inferosuperior articulation, and angular articulation relative to the Z-axis corresponds to medial rotational articulation.

As shown in FIG. 2, the first bone anchor 12 includes a body 30 and a post 32. The body 30 and the post 32 are optionally adapted to assist with securing the first bone anchor 12 directly to a scapula (not shown). For example, the first bone anchor 12 is optionally adapted to be secured to boney structures forming a glenoid cavity of the scapula. As shown, the body 30 includes one or more apertures 34 for receiving a fastener or fasteners (e.g., bone screws) for securing the body 30 to the scapula or other structure. The body 30 also defines an upper, shoulder portion 36 that is formed as a substantially flat plate and a lower, insert portion 38 that is reduced in diameter relative to the shoulder portion 36 and is formed as a hollow cylinder. In some embodiments, the bone anchor 12 is formed of titanium, for example, although other materials are contemplated.

In some embodiments, the post 32 is adapted to be secured directly to the scapula (e.g., including male threads or an appropriate geometry for assisting in attaching the first bone anchor 12 to the boney structures of the scapula). In other embodiments, the body 30 and/or the post 32 are adapted to interface with a secondary anchoring device (not shown) for securing the first bone anchor 12 to the scapula or other suitable structure, such as the secondary anchoring devices described in U.S. application Ser. No. 12/765,347, “Joint Prosthesis Attachment System, Device, and Method,” filed Apr. 22, 2010, the entire contents of which are incorporated herein by reference.

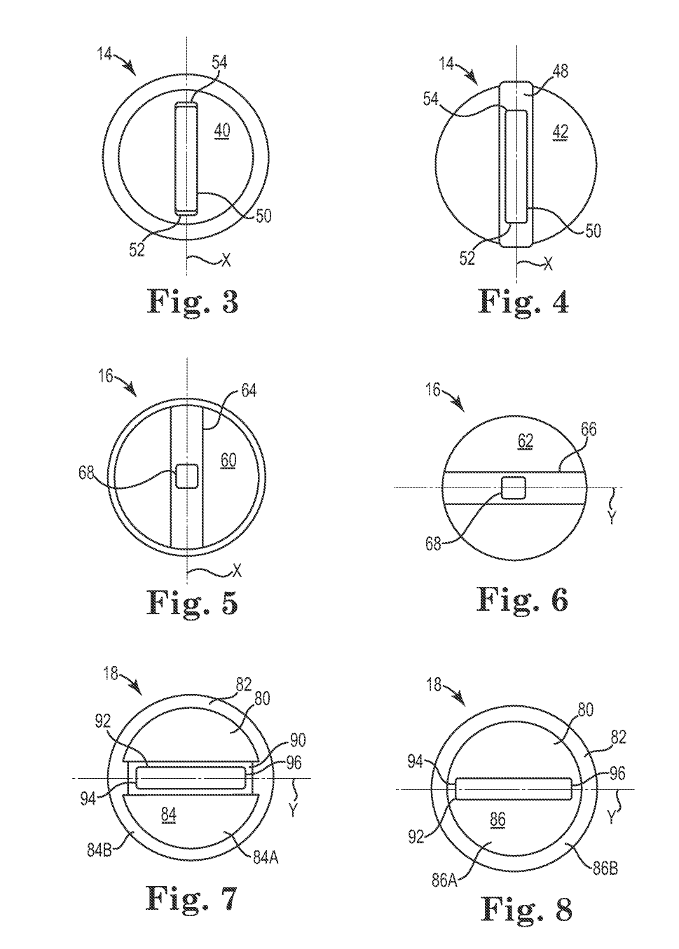

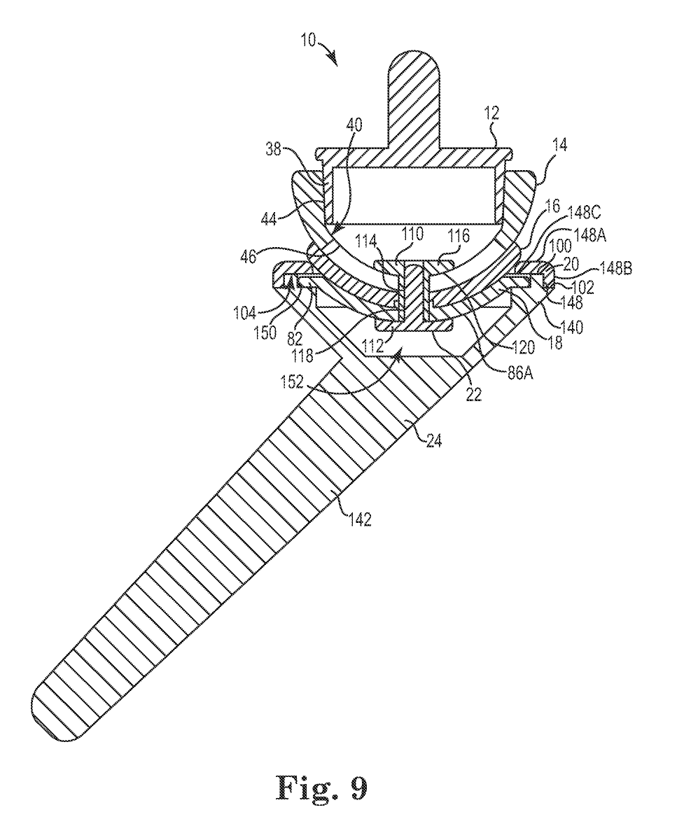

FIGS. 3 and 4 show the first articulation component 14 from a top view and a bottom view, respectively. As shown, the first articulation component 14 is a substantially hollow bowl, or is substantially cup-shaped, the first articulation component 14 having an inner surface 40 and an outer surface 42, the inner surface 40 being described as a first articulation surface and the outer surface 42 being described as a second articulation surface of the prosthesis 10. The outer surface 42 is substantially smooth overall and adapted for repeated articulation. As shown in the cross-sectional view of FIG. 9, the inner surface 40 has an upper portion 44 that is substantially cylindrical and a lower portion 46 that is substantially concave, where the upper portion 44 is adapted to form a complementary fit with the insert portion 38 of the first bone anchor 12.

In some embodiments, the upper portion 44 and the insert portion 38 are secured together using an interference or frictional fit, detents, fasteners, adhesives, combinations thereof, or other fastening means. As shown in FIG. 4, the outer surface 42 forms a track 48 (also described as a projection, a tenon, or a rail), that extends through an arcuate path diametrically (e.g., along a centerline or diameter of the first articulation component 14) across the first articulation component 14 in the X-axis direction, although the track 48 is also optionally comprised of one or more projections that extend along one or more parallel chords of the component 16. In some embodiments, the track 48 has a substantially rectangular cross-section, although a variety of shapes, such as dovetail cross-sections, are contemplated.

The first articulation component 14 is optionally formed of cobalt-chrome alloy and/or other suitable materials having low friction and/or wear characteristics for the outer surface 42, such as PTFE. Though some specific examples have been provided, a variety of materials are contemplated.

As shown in FIGS. 3 and 4, the first articulation component 14 also includes a slot 50, or opening, that is centered on the first articulation component 14, for example at an apex of the first articulation component 14. In some embodiments, the slot 50 is formed through the track 48, from the inner surface 40 to the outer surface 42, and has an arc length that is substantially shorter than that of the track 48. The slot 50 extends from a first end 52 to a second end 54, the first and second ends 52, 54 defining limits, or a range of movement, of the prosthesis 10 in a first direction, such as angular articulation relative to the Y-axis, or parallel to the X-Y plane, also described as a change in pitch.

FIGS. 5 and 6 show the second articulation component 16 from a top view and a bottom view, respectively. In some embodiments, the second articulation component 16 is a substantially hollow bowl, or is substantially cup-shaped, the second articulation component 16 having an inner surface 60 (also described as a third articulation surface) and an outer surface 62 (also described as a fourth articulation surface). The inner surface 60 is substantially concave and forms a first recess 64 and the second outer surface 62 is substantially convex and defines a second recess 66, each of the recesses 64, 66 also being described as guides, mortises, or channels. The second articulation component 16 also has an aperture 68 through the second articulation component 16 from the inner surface 60 to the outer surface 62. In some embodiments, the inner and outer surfaces 60, 62 are generally smooth, being adapted for repeated articulation.

As shown in FIGS. 2 and 5, the first recess 64 extends through an arcuate path diametrically (e.g., along a centerline or diameter of the second articulation component 16) across the inner surface 60 in the X-axis direction. In other embodiments, the second articulation component 16 includes one or more parallel recesses extending along one or more parallel chords of the component 16 in the X-axis direction. The first recess 64 has a substantially rectangular cross-section that is complementary to that of the track 48 of the first articulation component 14, although a variety of cross-sections, such as complementary, interlocking dovetail cross-sections, are also contemplated.

As shown in FIG. 6, the second recess 66 extends through an arcuate path diametrically (e.g., along a centerline or diameter of the second articulation component 16) across the outer surface 62 of the second articulation component 16 in the Y-axis direction. As shown, the second recess 66 extends in a substantially orthogonal direction to the first recess 64 of the second articulation component 16. In other embodiments, the second articulation component includes one or more parallel recesses extending along one or more parallel chords of the component 16 in the Y-axis direction. As shown, the second recess 66 has a substantially rectangular cross-section that is complementary to a track feature of the third articulation component 18, although a variety of cross-sections, such as complementary, interlocking dovetail cross-sections, are also contemplated.

As shown in FIGS. 5 and 6, the aperture 68 is formed through the second articulation component 16, from the inner surface 60 to the outer surface 62. In some embodiments, the aperture 68 is optionally positioned at an apex or center of the second articulation component 16 and has a substantially non-circular cross-section, such as a generally square cross-section, for example.

The second articulation component 16 is optionally formed of ultra-high-molecular-weight-polyethylene (UHMWPE) or other suitable materials having low friction and/or wear characteristics for the inner and outer surfaces 60, 62, such as PTFE. Though some specific examples have been provided, a variety of materials are contemplated.

FIGS. 7 and 8 show the third articulation component 18 from a top view and a bottom view, respectively. As shown, the third articulation component 18 includes a central portion 80 that is shaped as a substantially hollow bowl, or is substantially cup-shaped and a perimeter portion 82 that is shaped as a substantially flat rim extending from the central portion 80. The third articulation component 18 also has an inner surface 84 and an outer surface 86. In some embodiments, the inner and outer surfaces 84, 86 are substantially smooth overall and adapted for repeated articulation. As subsequently described, the inner surface 84 is adapted to engage and articulate with the outer surface 62 of the second articulation component 16 to define a second articulation interface.

In some embodiments, at the central portion 80, the inner surface 84 is substantially concave and defines a fifth articulation surface 84A. At the perimeter portion 82, the inner surface 84 is substantially flat, or planar, and defines a sixth articulation surface 84B. And, at the central portion 80, the outer surface 86 is substantially convex and defines a seventh articulation surface 86A and, at the perimeter portion 82, the outer surface 86 is substantially flat, or planar and defines a eighth articulation surface 86B.

As shown in FIG. 7, the inner surface 84 of the third articulation component 18 forms a track 90 (also described as a projection, a strip, a tenon, or a rail), that extends through an arcuate path diametrically (e.g., along a centerline or diameter) across the third articulation component 18 in the Y-axis direction, although the track 90 is also optionally comprised of one or more projections that extend along one or more parallel chords of the component 18. In some embodiments, the track 90 has a substantially rectangular cross-section, although a variety of shapes, such as dovetail cross-sections, are contemplated.

As shown in FIGS. 7 and 8, the third articulation component 18 also includes a slot 92, or opening, that is centered on the third articulation component 18, for example at an apex of the third articulation component 18. In some embodiments, the slot 92 is formed through the track 90, from the inner surface 84 to the outer surface 86, and has an arc length that is substantially shorter than that of the track 90. The slot 92 extends from a first end 94 to a second end 96, the first and second ends 94, 96 defining a limit, or range of movement, of the prosthesis 10 in a second direction that is angularly offset from the first direction in which the slot 50 of the first articulation component 14 extends. For example, in some embodiments, the second direction is orthogonal to the first direction and corresponds to angular articulation relative to the X-axis, or in the Y-Z plane, also described as a change in yaw.

The third articulation component 18 is optionally formed of cobalt-chrome alloy or other suitable materials having low friction and/or wear characteristics for the inner and outer surfaces 84, 86, such as PTFE. Though some specific examples have been provided, a variety of materials are contemplated.

FIG. 9 is a cross-section of the prosthesis 10 shown in an assembled state, according to some embodiments. As shown in FIGS. 2 and 9, the locking ring 20 is ring-shaped, having a substantially circular profile with an open interior, and includes an upper, cap portion 100 (also described as a lid or a retainer portion) and a lower collar portion 102 (also described as a lip or a shoulder portion). The cap portion 100 has an inner surface 104 and is adapted to retain the perimeter portion 82 of the third articulation component 18 in a seated position with the second bone anchor 24. In some embodiments, an inner surface 104 the cap portion 100 defines a ninth articulation surface of the prosthesis 10, the inner surface 104 being adapted to slide against the perimeter portion 82 of the third articulation component 18.

The collar portion 102 is adapted to fit with the second bone anchor 24 for securing the locking ring 20 to the second bone anchor 24. In some embodiments, the collar portion 102 includes female threads for securing the collar portion 102 to the second bone anchor 24. In other embodiments, the collar portion 102 additionally or alternative is adapted to be secured to the second bone anchor 24 using an adhesive or other fixation means. In some embodiments, the locking ring 20 is formed of titanium, although a variety of materials are contemplated.

As shown in FIGS. 2 and 9, the peg 22 includes a female connector 110 and a male connector 112. In some embodiments, the male and female connectors 110, 112 are formed of cobalt-chrome with a UHMWPE liner for suitable wear and/or low friction characteristics, although other materials are contemplated. The female connector 110 includes body 114 and a cap 116. The body 114 is substantially elongate, hollow, and has a non-circular outer cross-section according to some embodiments (e.g., substantially square-shaped). The body 114 also optionally defines an internal lumen 118 (FIG. 9) that is cylindrical in shape. The body 114 is generally adapted to be received through the three articulation components 14, 16, 18—through the slot 50, the aperture 68 (FIGS. 5 and 6), and the slot 92. The cap 116 is substantially wider than the slot 50 and has a lower surface 120 (FIG. 9), also described as a tenth articulation surface, for sliding against the inner surface 40 of the first articulation component 14.

As shown in FIG. 2, the male connector 112 includes a post 126 and a cap 128. The post 126 is substantially complementary in shape to the cross-section of the internal lumen 118 of the female connector 112 (e.g., substantially cylindrical). In some embodiments, the post 126 is adapted to be received in the body 114 of the female connector 110 in a complementary fit to secure the male and female connectors 110, 112 together. If desired, a fastener (not show), such as a screw, is driven through the cap 116 of the female connector 110 into the post 126 to secure the peg 22 together. In other embodiments, the male and female connectors 110, 112 are additionally or alternatively secured together with adhesive or using other fastening means. As shown in FIG. 9, the cap 128 is larger than the slot 92 of the third articulation component 18 such that the cap 128 is adapted to ride on the seventh articulation surface 86A of the third articulation component 18.

As shown in FIGS. 2 and 9, the second bone anchor 24 includes a head portion 140 and a stem portion 142. In some embodiments, the second bone anchor 24 is formed of titanium, although a variety of materials are contemplated. The head portion 140 and/or the stem portion 142 is optionally adapted to assist with securing the second bone anchor 24 directly to a humerus. For example, the stem portion 142 of the second bone anchor 14 is optionally substantially elongate and adapted to be secured within the proximal medullary canal of the humerus, though the second bone anchor 24 is optionally adapted to be secured to other boney structures, such as the femur in cases where the prosthesis 10 is adapted for hip replacement, for example.

The head portion 140 is substantially conical in shape and forms an outer flange 148, a support surface 150 (also described as an eleventh articulation surface), and a recessed pocket 152. In some embodiments, the head portion 140 is adapted to serve as a fourth articulation component and is rotatable with respect to the third articulation component 18 as subsequently described.

The outer flange 148 is substantially vertically oriented relative to the support surface 150. As shown, the outer flange 148 includes a top wall 148A adapted to support the cap portion 100 of the locking ring 20 and an outer wall 148B adapted to be secured to the collar portion 102 of the locking ring 20. The outer flange 148 also defines an inner wall 148C which helps retain the perimeter portion 82 of the third articulation component 18 in the head portion 140 and against which an edge of the perimeter portion 82 optionally slides. The support surface 150 is adapted to slidingly support and engage the eighth articulation surface 86B on the perimeter portion 82 of the third articulation component 18. The recessed pocket 152 is adapted to receive portions of the first, second, and third articulation components 12, 14, 16, as well as the peg 22, such that the components are free to angularly articulate as desired.

Assembly of the prosthesis 10 from the unassembled state of FIG. 2 to the assembled state includes mating the track 48 of the first articulation component 14 with the first recess 64 of the second articulation component 16 such that the first articulation component 14 is able to articulate relative to the Y-axis while being substantially constrained from angular articulation relative to the X- or Z-axes. Upon mating the track 48 and recess 64, the inner surface 60 of the second articulation component 16 slides against the outer surface 42 of the first articulation component 14 to define a first articulation interface between the surfaces 42, 60, where the second articulation component 16 provides a bearing surface or acts as a bushing for repeated articulation with the first articulation component 14. Thus, the track 48 and the first recess 64 optionally provide means for limiting angular articulation of the first articulation component 14, which is in sliding contact with the second articulation component 16, to changes in pitch.

The track 90 of the third articulation component 18 is mated with the second recess 66 of the second articulation component 16 such that the third articulation component 18 is able to articulate with the second articulation component 14 relative to the X-axis while being substantially constrained from articulating in rotational or other directions relative to the Y- or Z-axes. In some embodiments, upon mating the track 90 and second recess 66, the outer surface 62 of the second articulation component 16 engages and slides against the inner surface 84 of the third articulation component 18 to define a second articulation interface between the surfaces 62, 84 where the second articulation component 16 provides a bearing surface or acts as a bushing for repeated articulation with the third articulation component 18. Thus, the track 90 and second recess 66 optionally provide means for limiting angular articulation of the third articulation component 18, which is in sliding contact with the second articulation component 16, to changes in yaw.

The first, second, and third components are secured together with the peg 22 by inserting the female connector 110 through the three articulation components 14, 16, 18—through the slot 50 (FIG. 3), the aperture 68 (FIG. 5), and the slot 92. The male connector 112 is inserted into the female connector 110 and secured in place. In some embodiments, the non-circular cross-sections of the body 114 of the female connector 110, the slots 50, 92, and the aperture 68 help ensure that the three components 14, 16, 18 do not articulate relative to the Z-axis, or change in roll, with respect to one another while still leaving the components 14, 16, 18 free to angularly articulate relative to Y- and X-axes, respectively.

In some embodiments, the three articulation components 14, 16, 18 are secured between the first and second bone anchors 12, 24 such that the first and second bone anchors 12, 24 are able to rotate, or angulate relative to the Z-axis as well as angulate relative to the X- and Y-axes as described, where the second bone anchor 24 is optionally described as fourth articulation component and the first bone anchor 12 is optionally described as a fifth articulation component. For example, in some embodiments, implantation of the prosthesis 10 includes securing the first bone anchor 12 to a first bone (not shown) such as a scapula. The first bone anchor 12 is optionally secured directly to the first bone (e.g., using bone screws) or using a secondary anchoring device, such as those previously described. The first bone anchor 12 is secured to the first articulation component 14 by positioning the insert portion 38 of the first bone anchor 12 into the upper portion 44 of the first articulation component such that the first bone anchor 12 is fixed to, and moves with, the first articulation component 14 as a single piece. In some embodiments, the insert portion 38 and the upper portion 44 are secured together with the help of adhesives and/or mechanical fasteners (not shown).

In some embodiments, the second bone anchor 24 is secured to a second bone (not shown), such as a humerus, using known techniques. For example, in some embodiments, the stem portion 142 of the second bone anchor 24 is secured in a proximal medullary cavity of a humerus.

As shown in FIG. 9, the head portion 140 of the second bone anchor 24 is secured to the third articulation component 18 by receiving the perimeter portion 82 of the third articulation component against the support surface 150 of the second bone anchor 24. The locking ring 20 is secured over the perimeter portion 82 and onto the head portion 140 of the second bone anchor 24 such that the third articulation component 18 is free to rotate with respect to the second bone anchor 24, the perimeter portion 82 and the support surface 150 engaging to form a third articulation interface and the perimeter portion 82 and the locking ring 20 engaging to form a fourth articulation interface of the prosthesis 10. Thus, the third and fourth articulation interfaces optionally provide means for allowing changes in roll between the first bone anchor 12 and the second bone anchor 24.

Upon securing the articulation components 14, 16, 18 to the bone anchors 12, 24, the prosthesis 10 is linked, forming a fixed assembly that limits subluxation between the first and second bone anchors 12, 24 and is able to freely articulate. For example, in some embodiments, the prosthesis is adapted such that substantially no subluxation is allowed between the first and second bone anchors 12, 24.

Articulation of the prosthesis 10 includes articulating the first and second articulation components 14, 16 relative to one another such that the first articulation component 14 angulates and shifts laterally relative to the second articulation component 16 along a first arcuate path extending in the X-Z plane. In particular, the first component 14 is guided in the X-Z plane as the track 48 rides within the first recess 64 of the second articulation component 16 such that the first articulation component 14 only articulates in the X-Z plane relative to the second articulation component 16, or only changes in pitch, and is substantially constrained from articulating in other directions relative to the second articulation component 16. The peg 22 rides in the slot 50 in the first articulation component with the first and second ends 52, 54 of the slot 50 serving as stops, or limits to the range of travel of the prosthesis in the X-Z plane. Substantially all of the X-Z plane articulation of the prosthesis 10 occurs at the first articulation interface between the first and second articulation components 14, 16, including the track 48 and the first recess 64, such that the inner surface 60 of the second articulation component 16 is only exposed to wear in one direction, the X-axis direction, rather than all directions as would otherwise be the case in a traditional ball-and-socket joint, helping increase wear life of the prosthesis 10.

In some embodiments, the third articulation component 18 is articulated relative to the second articulation component 16 such that the third articulation component 18 angulates and shifts laterally relative to the second articulation component 16 along a second arcuate path extending parallel to the Y-Z plane. In particular, the third articulation component 18 is guided in the Y-Z plane as the track 90 rides within the second recess 66 of the second articulation component 16 such that the third articulation component 18 only articulates in the Y-Z plane relative to the second articulation component 16, or only changes in yaw, and is substantially constrained from articulating in other directions relative to the second articulation component 16.

In some embodiments, the peg 22 rides in the slot 92 in the third articulation component 18 with the first and second ends 94, 96 of the slot 92 serving as stops, or limits in the range of travel of the prosthesis in the Y-Z plane. Thus, according to some embodiments, substantially all of the Y-Z plane articulation of the prosthesis 10 occurs at the first articulation interface between the third and second articulation components 18, 16, including the track 90 and the second recess 66, such that the outer surface 62 of the second articulation component 16 is only exposed to wear in one direction, the Y-axis direction, rather than all directions as would otherwise be the case in a traditional ball-and-socket joint, also helping increase wear life of the prosthesis 10.

In some embodiments, the third articulation component 18 is articulated relative to the head portion 140 of the second bone anchor 24, also described as a fourth articulation component, such that the third articulation component rotates, or angulates, in the X-Y plane relative to the Z-axis. In particular, the perimeter portion 82 of the third articulation component 18 is maintained between, and engages, the locking ring 20 and the support surface 150 at third and fourth articulation interfaces such that the third articulation component 18 only articulates in the X-Y plane, or changes in roll, relative to the head portion 140 and is substantially constrained from articulating in other directions relative to the head portion 140. In some embodiments, limits (not shown) such as slots or guides are provided to limit the range of travel of the prosthesis in the X-Y plane, or to limit roll of the prosthesis 10. Thus, according to some embodiments, substantially all of the X-Y plane articulation of the prosthesis 10 occurs at the third and fourth articulation interfaces between the third articulation component 18, the head portion 140, and the locking ring 20 such that the perimeter portion 82 of the third articulation component 18 is only exposed to wear in the rotational direction, rather than all directions as would otherwise be the case in a traditional ball-and-socket joint, also helping increase wear life of the prosthesis 10.

The three degrees of freedom (X-Z plane, Y-Z plane, and X-Y plane) help the prosthesis 10 act as a virtual ball-and-socket joint, with comparable mobility and a substantially medialized center of rotation, while maintaining the articulation components in a linked, substantially non-subluxating configuration. For example, the prosthesis 10 is optionally adapted to facilitate articulation between the humerus and the scapula through a natural range of motion, including flexion, extension, adduction, abduction, and rotation.

While certain components have been referred to as forming a track and others a recess for receiving the track according to various embodiments, it should be understood that in other embodiments the track(s) and recess(es) are optionally reversed on the components.

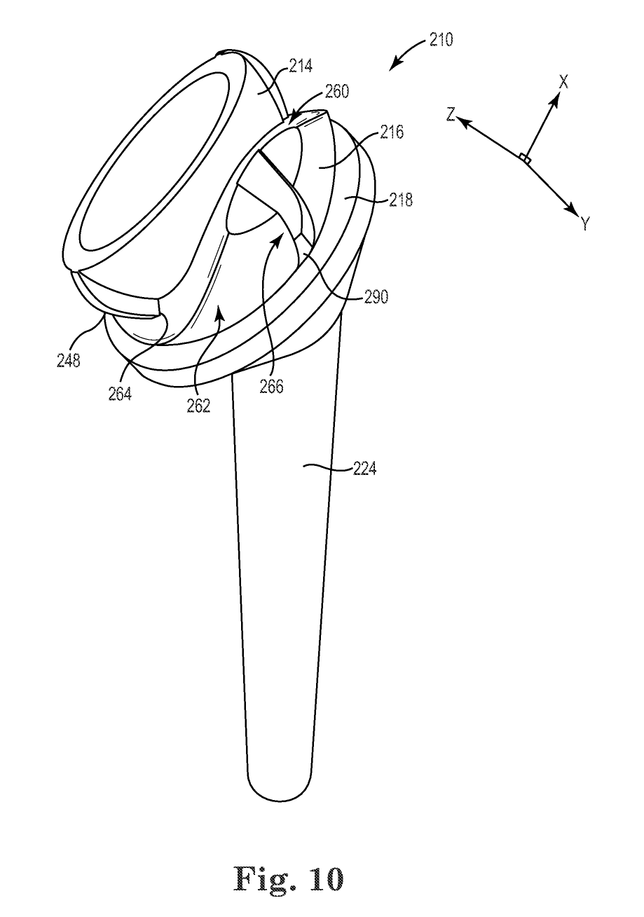

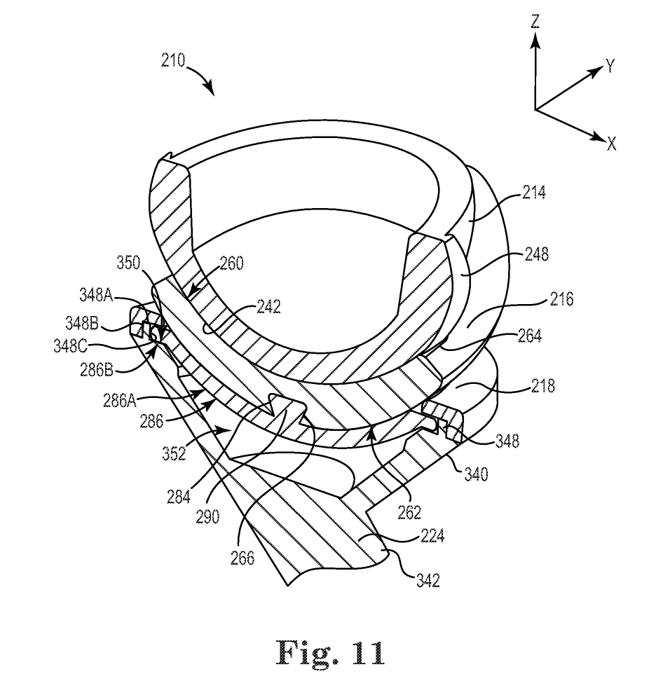

FIG. 10 is a perspective view and FIG. 11 is a cut away view of another prosthesis 210, according to some embodiments. As shown, the prosthesis 210 includes, a first articulation component 214 (also described as a glenosphere), a second articulation component 216 (also described as a liner or a disk), a third articulation component 218 (also described as a rotational plate), and a second bone anchor 224 (also described as a stem). Though not shown, the prosthesis 210 also includes a base plate (e.g., formed of titanium) a locking ring (e.g., formed of titanium) and a first bone anchor (e.g., formed of titanium) substantially similar to those of the prosthesis 10, according to some embodiments. Moreover, the prosthesis 210 optionally includes any of the features described in association with the prosthesis 10 and vice versa, as desired.

The prosthesis 210 is generally adapted as a virtual ball-and-socket joint, being able to articulate through a wide range of motion similar to that of a traditional ball-and-socket joint by supporting freedom of movement between in at least three coordinate directions, such as X-, Y-, and Z-axis angular articulation. For example, the prosthesis 210 optionally facilitates angular articulation relative to the X-axis (also described as pitch), or parallel to the Y-Z plane, relative to the Y-axis (also described as yaw), or parallel to the X-Z plane, and relative to the Z-axis (also described as roll), or parallel to the X-Y plane. In some embodiments, angular articulation relative to the X-axis corresponds to front-back motion or anteroposterior articulation, angular articulation relative to the Y-axis corresponds to up-down motion or inferosuperior IS articulation, and angular articulation relative to the Z-axis corresponds to medial rotational articulation.

FIG. 12 shows the first articulation component 214 from a perspective view. As shown, the first articulation component 214 is a substantially hollow bowl, or is substantially cup-shaped, the first articulation component 214 having an inner surface 240 and an outer surface 242, the inner surface 240 being described as a first articulation surface and the outer surface 242 being described as a second articulation surface of the prosthesis 210. The outer surface 242 is substantially smooth overall and adapted for repeated articulation. As shown, the inner surface 240 has an upper portion 244 that is substantially cylindrical and a lower portion 246 that is substantially concave, where the upper portion 244 is adapted to form a complementary fit with an insert portion of a bone anchor, such as the first bone anchor 12.

In some embodiments, the upper portion 244 is secured to an insert portion using an interference or frictional fit, detents, fasteners, adhesives, combinations thereof, or other fastening means. As shown, the outer surface 242 forms a track 248 (also described as a projection, a tenon, or a rail), that extends through an arcuate path diametrically (e.g., along a centerline or diameter of the first articulation component 14) across the first articulation component 214 in the X-axis direction, although the track 248 is also optionally comprised of one or more projections that extend along one or more parallel chords of the component 216. As shown, the track 248 has a dovetail shaped cross-section adapted to interlock with a complementary cross-section, although a variety of interlocking shapes (e.g., interlocking D-shapes, star-shapes, or others), are contemplated.

The first articulation component 214 is optionally formed of cobalt-chrome alloy or other suitable materials having low friction and/or wear characteristics for the outer surface 242, such as PTFE. Though some specific examples have been provided, a variety of materials are contemplated.

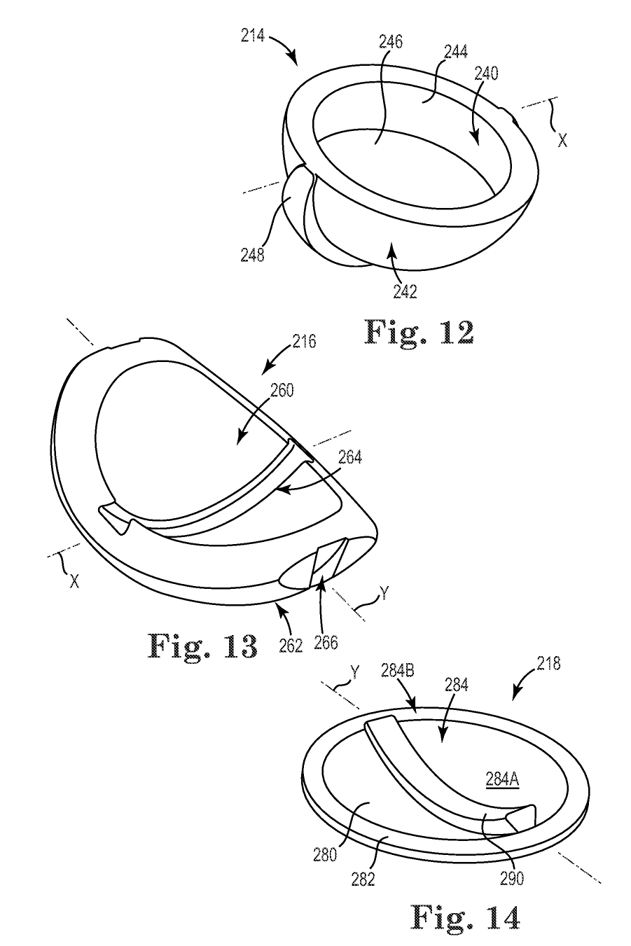

FIG. 13 shows the second articulation component 216 from a perspective view, according to some embodiments. As shown, the second articulation component 216 is a substantially hollow bowl, or is substantially cup-shaped, the second articulation component 216 having an inner surface 260 (also described as a third articulation surface) and an outer surface 262 (also described as a fourth articulation surface). The inner surface 260 is substantially concave and forms a first recess 264 and the second outer surface 262 is substantially convex and defines a second recess 266, each of the recesses also being described as guides or mortises. In some embodiments, the inner and outer surfaces 260, 262 are generally smooth, being adapted for repeated articulation.

As shown, the first recess 264 extends through an arcuate path diametrically (e.g., along a centerline or diameter of the second articulation component 216) across the inner surface 260 in the X-axis direction. In other embodiments, the second articulation component 216 includes one or more parallel recesses extending along one or more parallel chords of the component 216 in the X-axis direction. As shown, the first recess 264 has a substantially dovetail shaped cross-section that is complementary to that of the track 248 of the first articulation component 214, although a variety of interlocking cross-sections are contemplated.

In some embodiments, the second recess 266 extends through an arcuate path diametrically (e.g., along a centerline or diameter of the second articulation component 216) across the outer surface 262 of the second articulation component 216 in the Y-axis direction. The second recess 266 extends in a substantially orthogonal direction to the first recess 264 of the second articulation component 216. In other embodiments, the second articulation component 216 includes one or more parallel recesses extending along one or more parallel chords of the component 216 in the Y-axis direction. As shown, the second recess 266 has a substantially dovetail shaped cross-section that is complementary to a track feature of the third articulation component 218, although a variety of shapes are also contemplated.

The second articulation component 16 is optionally formed of UHMWPE or other suitable materials having low friction and/or wear characteristics for the inner and outer surfaces 260, 262, such as PTFE. Though some specific examples have been provided, a variety of materials are contemplated.

FIG. 14 shows the third articulation component 218 from a top view and a bottom view, respectively. As shown, the third articulation component 218 includes a central portion 280 that is shaped as a substantially hollow bowl, or is substantially cup-shaped and a perimeter portion 282 that is shaped as a substantially flat rim extending from the central portion 280. The third articulation component 218 also has an inner surface 284 and an outer surface 286 (FIG. 11). The inner and outer surfaces 284, 286 are substantially smooth overall and adapted for repeated articulation. As subsequently described, the inner surface 284 is adapted to engage and articulate with the outer surface 262 of the second articulation component 216 to define a second articulation interface.

As shown in FIG. 14, at the central portion 280, the inner surface 284 is substantially concave and defines a fifth articulation surface 284A. At the perimeter portion 282, the inner surface 284 is substantially flat, or planar, and defines a sixth articulation surface 284B. As shown in FIG. 11, at the central portion 280, the outer surface 286 defines a seventh articulation surface 286A and at the perimeter portion 282, the outer surface 286 is substantially flat, or planar, and defines a eighth articulation surface 286B.

As shown in FIG. 14, the inner surface 284 of the third articulation component 218 forms a track 290 (also described as a projection, a strip, a tenon, or a rail), that extends through an arcuate path diametrically (e.g., along a centerline or diameter of the third articulation component 218) across the third articulation component 218 in the Y-axis direction, although the track 290 is also optionally comprised of one or more projections that extend along one or more parallel chords of the component 218. In some embodiments, the track 290 has a substantially dovetail shaped cross-section, although a variety of shapes are contemplated.

The third articulation component 218 is optionally formed of cobalt-chrome alloy or other suitable materials having low friction and/or wear characteristics for the inner and outer surfaces 284, 286, such as PTFE. Though some specific examples have been provided, a variety of materials are contemplated.

As shown in FIG. 11, the second bone anchor 224 includes a head portion 340 and a stem portion 342. The head portion 340 and/or the stem portion 342 is optionally adapted to assist with securing the second bone anchor 224 directly to a humerus. For example, the stem portion 342 of the second bone anchor 224 is optionally substantially elongate and adapted to be secured within the proximal medullary canal of the humerus, though the second bone anchor 224 is optionally adapted to be secured to other boney structures, such as the femur in cases where the prosthesis 210 is adapted for hip replacement, for example. In some embodiments, the second bone anchor 224 is formed of titanium, although a variety of materials are contemplated.

The head portion 340 is substantially conical in shape and forms an outer flange 348, a support surface 350 (also described as an eleventh articulation surface), and a recessed pocket 352. In some embodiments, the head portion 340 is adapted to serve as a fourth articulation component and is rotatable with respect to the third articulation component as subsequently described.

The outer flange 348 is substantially vertically oriented relative to the support surface 350. As shown, the outer flange 348 includes a top wall 348A adapted to support a cap portion of a locking ring, such as the locking ring 20, and an outer wall 348B adapted to be secured to a collar portion of a locking ring, such as the locking ring 20. The outer flange 348 also defines an inner wall 348C which helps retain the perimeter portion 282 of the third articulation component 218 in the head portion 340 and against which an edge of the perimeter portion 282 optionally slides. The support surface 350 is adapted to slidingly support and engage the eighth articulation surface 286B on the perimeter portion 282 of the third articulation component 218. The recessed pocket 352 is adapted to receive portions of the first, second, and third articulation components 212, 214, 216, such that the components are free to angularly articulate as desired.

Assembly of the prosthesis 210 from an unassembled state to the assembled state shown in FIG. 11 includes mating the track 248 of the first articulation component 214 with the first recess 264 of the second articulation component 216 such that the first articulation component 214 is able to articulate relative to the Y-axis while being substantially constrained from angular articulation relative to the X- or Z-axes. In some embodiments, upon mating the track 248 and recess 264, the inner surface 260 of the second articulation component 216 slides against the outer surface 242 of the first articulation component 214 to define a first articulation interface between the surfaces 242, 260, where the second articulation component 216 provides a bearing surface or acts as a bushing for repeated articulation with the first articulation component 214. The interlocking shapes of the track 248 and the recess 264 links the first and second components 214, 216 such that subluxation, or separation between the first and second articulation components 214, 216 is substantially prevented, or otherwise limited. Thus, the track 248 and the first recess 264 optionally provide means for limiting angular articulation of the first articulation component 214, which is in sliding contact with the second articulation component 216, to changes in pitch.

The track 290 of the third articulation component 18 is mated with the second recess 266 of the second articulation component 216 such that the second and third articulation components 216, 218 are able to articulate relative to the X-axis while being substantially constrained from articulating in rotational or other directions relative to the Y- or Z-axes. The interlocking shapes of the track 290 and the recess 266 links the second and third components 216, 218 such that subluxation, or separation between the second and third articulation components 216, 218 is substantially prevented, or limited. In some embodiments, upon mating the track 290 and second recess 266, the outer surface 262 of the second articulation component 216 engages and slides against the inner surface 284 of the third articulation component 218 to define a second articulation interface between the surfaces 262, 284 where the second articulation component 216 provides a bearing surface or acts as a bushing for repeated articulation with the third articulation component 218. Thus, the track 290 and second recess 266 optionally provide means for limiting angular articulation of the third articulation component 218, which is in sliding contact with the second articulation component 216, to changes in yaw.

In some embodiments, the three articulation components 214, 216, 218 are secured between a first bone anchor, such as the bone anchor 12, and the second bone anchor 224 such that the bone anchors are able to change in roll, or angulate relative to the Z-axis, or in the X-Y plane, as well as change in relative pitch and yaw. For example, in some embodiments, implantation of the prosthesis 210 includes securing a first bone anchor (e.g., the first bone anchor 12) to a first bone (not shown) such as a scapula. The first bone anchor is optionally secured directly to the first bone (e.g., using bone screws) or using a secondary anchoring device, such as those previously described. The first bone anchor is secured to the first articulation component 214 by positioning the insert portion of the first bone anchor into the upper portion 244 of the first articulation component 214. In some embodiments, the insert portion and the upper portion 244 are secured together with the help of adhesives and/or mechanical fasteners (not shown).

In some embodiments, the second bone anchor 224 is secured to a second bone (not shown), such as a humerus, using known techniques. For example, in some embodiments, the stem portion 342 of the second bone anchor 224 is secured in a proximal medullary cavity of a humerus. In other embodiments, the bone anchors are secured between another set of bones, such as between a femur and a pelvis to serve as an artificial hip.

As shown in FIG. 11, the head portion 340 of the second bone anchor 224 is secured to the third articulation component 218 by receiving the perimeter portion 282 of the third articulation component 218 against the support surface 350 of the second bone anchor 224. The locking ring (not shown) is secured over the perimeter portion 282 and onto the head portion 340 of the second bone anchor 224 such that the third articulation component 218 is free to rotate with respect to the second bone anchor 224, the perimeter portion 282 and the support surface 350 engaging to form a third articulation interface and the perimeter portion 282 and the locking ring engaging to form a fourth articulation interface of the prosthesis 310. Thus, the third and fourth articulation interfaces optionally provide means for allowing changes in roll between the first bone anchor and the second bone anchor 224.

Upon securing the articulation components 214, 216, 218 to the bone anchors, the entire prosthesis 210 is linked, forming a fixed assembly that limits subluxation between the bone anchors and is able to freely articulate. For example, in some embodiments, the prosthesis 210 is adapted such that substantially no subluxation is allowed between the bone anchors, and thus between the bones to which they are secured (e.g., the humerus and scapula).

Articulation of the prosthesis 210 includes articulating the first and second articulation components 214, 216 relative to one another such that the first articulation component 214 angulates and shifts laterally relative to the second articulation component 216 along a first arcuate path extending in the X-Z plane. In particular, the first component 214 is guided in the X-Z plane as the track 248 rides within the first recess 264 of the second articulation component 216. The track 248 only permits the first articulation component 214 to articulate in the X-Z plane relative to the second articulation component 216, or only to change in pitch, and substantially constrains articulation between the first and second articulation components 214, 216 in other directions.

In some embodiments, substantially all of the X-Z plane articulation of the prosthesis 210 occurs at the first articulation interface between the first and second articulation components 214, 216, including the track 248 and the first recess 264, such that the inner surface 260 of the second articulation component 216 is only exposed to wear in one direction, the X-axis direction, rather than all directions as would otherwise be the case in a traditional ball-and-socket joint, helping increase wear life of the prosthesis 210.

In some embodiments, the third articulation component 218 is articulated relative to the second articulation component 216 such that the third articulation component 218 angulates and shifts laterally relative to the second articulation component 216 along a second arcuate path extending parallel to the Y-Z plane. In particular, the third articulation component 218 is guided in the Y-Z plane as the track 290 rides within the second recess 266 of the second articulation component 216 such that the third articulation component 218 only articulates in the Y-Z plane relative to the second articulation component 16, or only changes in yaw, and is substantially constrained from articulating in other directions relative to the second articulation component 216.

According to some embodiments, substantially all of the Y-Z plane articulation of the prosthesis 210 occurs at the first articulation interface between the third and second articulation components 218, 216, including the track 290 and second recess 266, such that the outer surface 262 of the second articulation component 216 is only exposed to wear in one direction, the Y-axis direction, rather than all directions as would otherwise be the case in a traditional ball-and-socket joint, also helping increase wear life of the prosthesis 210.

In some embodiments, the third articulation component 218 is articulated relative to the head portion 340 of the second bone anchor 224, also described as a fourth articulation component, such that the third articulation component rotates, or angulates, in the X-Y plane relative to the Z-axis. In particular, the perimeter portion 282 of the third articulation component 218 is maintained between, and engages, the locking ring (not shown) and the support surface 350 at third and fourth articulation interfaces such that the third articulation component 218 only articulates in the X-Y plane, or changes in roll, relative to the head portion 340 and is substantially constrained from articulating in other directions relative to the head portion 340.

According to some embodiments, substantially all of the X-Y plane articulation of the prosthesis 210 occurs at the third and fourth articulation interfaces between the third articulation component 218, the head portion 340, and the locking ring, such that the perimeter portion 282 of the third articulation component 218 is only exposed to wear in the rotational direction, rather than all directions as would otherwise be the case in a traditional ball-and-socket joint, also helping increase wear life of the prosthesis 210.

The three degrees of freedom (X-Z plane, Y-Z plane, and X-Y plane) help the prosthesis 210 act as a virtual ball-and-socket joint, with comparable mobility and a substantially medialized center of rotation, while maintaining the articulation components in a linked, substantially non-subluxating configuration. For example, the prosthesis 210 is optionally adapted to facilitate articulation between the humerus and the scapula through a natural range of motion, including flexion, extension, adduction, abduction, and rotation.

While certain components have been referred to as forming a track and others a recess for receiving the track according to various embodiments, it should be understood that in other embodiments the track(s) and recess(es) are optionally reversed on the components. For example, the track 48 is optionally formed on the second articulation component 16 with the corresponding recess 64 on the first articulation component 14, and so forth.

Various embodiments and features thereof have been described with reference to relational terms. Unless context specifically dictates otherwise, the terms “first,” “second,” “third,” etc. used with reference to various features are not intended to require a particular order, but are used in a general sense to designate the different features for description purposes. Similarly, the terms “upper,” “lower,” “front,” “back,” “vertical,” “horizontal,” etc. are not intended to be limiting in nature, but are instead used to provide relative orientation between features being described.

Various modifications, permutations, and additions can be made to the exemplary embodiments and aspects of the embodiments discussed without departing from the scope of the present invention. For example, while the embodiments described above refer to particular features, the scope of this invention also includes embodiments having different combinations of features and embodiments that do not include all of the features. Accordingly, the scope of the present invention is intended to embrace all such alternatives, modifications, permutations, and variations as fall within the scope of the claims, together with all equivalents thereof.

The following is claimed:

1. A joint prosthesis adapted to be secured to a first bone and a second bone for facilitating relative articulation between the first and second bones, the joint prosthesis comprising:
first articulation component defining a first articulation surface that is substantially convex;
a second articulation component defining a second articulation surface that is substantially concave and a third articulation surface that is substantially convex, the first articulation component being engaged with the second articulation component such that the first articulation surface of the first articulation component articulates with the second articulation surface of the second articulation component; and
a third articulation component defining a fourth articulation surface that is substantially concave, the third articulation component being engaged with the second articulation component such that the third articulation surface of the second articulation component articulates with the fourth articulation surface of the third articulation component.

2. The joint prosthesis of claim 1, wherein the first articulation component is substantially limited in angulation relative to the second articulation component within a first plane and the third articulation component is substantially limited in angulation relative to the first articulation component within a second plane that is angularly offset from the first plane.

3. The joint prosthesis of claim 1, wherein the first, second, and third articulation components are secured together in a linked configuration that limits subluxation between the first, second, and third articulation components.

4. The joint prosthesis of claim 1, wherein the first and second planes are offset by 90 degrees.

5. The joint prosthesis of claim 1, wherein the first and third articulation components are secured relative to one another such that the first articulation component is limited from rotational angulation relative to the third articulation component.

6. The joint prosthesis of claim 1, further comprising a fourth articulation component rotatably secured relative to the third articulation component such that the first articulation component is free to change in angular rotation relative to the fourth articulation component.

7. The joint prosthesis of claim 1, wherein one of the first and second articulation surfaces defines a track and the other of the first and second articulation surfaces defines a channel for receiving the track, the track and channel mating to limit articulation of the first articulation component relative to the second articulation component.

8. The joint prosthesis of claim 1, wherein the first articulation component and the second articulation component are secured together by a dovetail joint that limits articulation of the first articulation component relative to the second articulation component.

9. The joint prosthesis of claim 1, wherein one of the third and fourth articulation surfaces defines a track and the other of the third and fourth articulation surfaces defines a channel for receiving the track, the track and channel mating to limit articulation of the third articulation component relative to the second articulation component.

10. The joint prosthesis of claim 1, wherein the second articulation component and the third articulation component are secured together by a dovetail joint that limits articulation of the third articulation component relative to the second articulation component.

11. The joint prosthesis of claim 1, wherein the first articulation surface is substantially smooth overall and adapted for repeated articulation.

12. The joint prosthesis of claim 1, further comprising a fourth articulation component adapted to be secured to a humerus and a fifth articulation component adapted to be secured to a scapula, the first, second, and third articulation components forming a virtual ball-and-socket joint between the fourth and fifth articulation components.

13. The joint prosthesis of claim 1, further comprising a fourth articulation component adapted to be secured to a femur and a fifth articulation component adapted to be secured to a pelvis, the first, second, and third articulation components forming a virtual ball-and-socket joint between the fifth and sixth articulation components.

14. The joint prosthesis of claim 1, wherein the first second and third articulation components are secured relative to one another by a peg that limits lateral angulation of the third articulation component relative to the first articulation component.

15. A virtual ball-and-socket prosthesis for replacing a joint between a first bone and a second bone, the prosthesis comprising:
means for limiting angular articulation of a first articulation component in sliding contact with a second articulation component to changes in pitch;
means for limiting angular articulation of a third articulation component in sliding contact with the second articulation component to changes in yaw;
first bone anchor means for securing the first articulation component to a first bone;
second bone anchor means for securing the third articulation component to a second bone; and
means for allowing changes in roll between the first bone anchor means and the second bone anchor means.

16. A virtual ball-and-socket prosthesis for replacing a natural joint between two bones, the prosthesis comprising a first bone anchor component, a second bone anchor component, and a plurality of articulation components that articulatably join the first and second bone anchor components, the plurality of articulation components defining a pitch bearing interface, a yaw bearing interface separate from the pitch bearing interface, and a roll bearing interface separate from both the pitch and yaw bearing interfaces, the plurality of articulation components being secured relative to one another such that articulation between the first and second bone anchors in pitch is borne by the pitch bearing surface, articulation between the first and second bone anchors in yaw is borne by the yaw bearing interface, and medial rotational articulation between the first and second bone anchors is borne by the rotational bearing interface.

17. A method of assembling an artificial joint between bones, the method comprising:
securing a first articulation component having a first articulation surface that is convex to a second articulation component having a second articulation surface that is concave such that the first articulation surface of the first articulation component is engaged with the second articulation surface of the second articulation component and the first articulation component is limited in angulation relative to the second articulation component to a first plane; and
securing a third articulation component defining a fourth articulation surface that is concave to the second articulation component such that a third articulation surface of the second articulation component that is convex is engaged with the fourth articulation surface of the third articulation component and the third articulation component is limited in lateral angulation relative to the first articulation component to a second plane that is angularly offset from the first plane.

18. The method of claim 17, further comprising linking the first, second, and third articulation components together to limit subluxation between the first, second, and third articulation components.

19. The method of claim 17, further comprising:
securing the first articulation component relative to a scapula and the third articulation component relative to a humerus; and
articulating the humerus relative to the scapula through a natural range of motion, including flexion, extension, adduction, abduction, and rotation of the artificial joint.

20. The method of claim 17, further comprising rotatably securing a fourth articulation component to the third articulation component such that the first articulation component is free to angulate rotationally relative to the fourth articulation component.

21. The method of claim 17, further comprising securing a fourth articulation component to the third articulation component and to a humerus.

22. The method of claim 17, further comprising inserting a pin through the first, second, and third articulation components to secure the first, second, and third articulation components relative to one another.

23. The method of claim 17, further comprising securing the first and second articulation components to one another using a dovetail joint that limits lateral angulation of the first articulation component relative to the second articulation component to the first plane.
